(19) United States
(12) Patent Application Publication
(10) Pub. No.: US 2026/0345380 A1
(43) Pub. Date: Nov. 5, 2026

(54) OPHTHALMIC DEVICE

(71) Applicant: Choryukai Medical Corporation, Kashima-shi Ibaraki (JP)

(72) Inventor: Hayate Nakagawa, Kashima-shi Ibaraki (JP)

(73) Assignee: Choryukai Medical Corporation, Kashima-shi Ibaraki (JP)

(21) Appl. No.: 19/657,193

(22) Filed: Apr. 24, 2026

Related U.S. Application Data
(63) Continuation of application No. PCT/JP2025/041042, filed on Nov. 25, 2025.

(30) Foreign Application Priority Data
Mar. 5, 2025 (JP) 2025-034504

(51) Int. Cl. A61F 9/00

(52) U.S. Cl. CPC A61F 9/0017

(57) ABSTRACT

An ophthalmic device for use in intrascleral fixation of an intraocular lens, the ophthalmic device being linear and having a longitudinal direction, includes: a sliding member configured to be slidable in the longitudinal direction, the sliding member being provided at a portion spaced apart by a predetermined distance from an end of the ophthalmic device to be punctured into an eye. An opening is formed between the end and the sliding member, the sliding member is configured to be slidable only in one direction in the longitudinal direction toward the end, and the opening is variable in size as the sliding member slides toward the end.

### CROSS-REFERENCE TO RELATED APPLICATIONS

This application is a continuation application of International Application No. PCT/JP2025/041042, filed on Nov. 25, 2025, which claims priority of Japanese Patent Application No. 2025-034504, filed on Mar. 5, 2025, the contents of which are hereby incorporated by reference in its entirety.

### TECHNICAL FIELD

This disclosure relates to an ophthalmic device. More specifically, this disclosure relates to an ophthalmic device used for intrascleral fixation of an intraocular lens.

### BACKGROUND

In cataract surgery, an artificial intraocular lens has been implanted in the eye to replace the removed lens conventionally. The intrascleral fixation technique, in which the support of the intraocular lens is implanted in the sclera, which forms the outer wall of the eyeball, has become a popular method of fixing the intraocular lens in the eye in recent years. In the intrascleral fixation technique, the support of the intraocular lens placed in the eye needs to be pulled out of the eye and fixed in place, and instruments for this purpose have been disclosed (e.g., Japanese Registered Patent No. 6502431).

### SUMMARY

An intraocular lens implanted in the eye may fall into the eyeball for some reason. In this case, the intraocular lens that has fallen is removed from the eyeball and a new intraocular lens is inserted; however, due to the strain on the eyeball, a method to reuse the original intraocular lens has been needed. However, the instruments described in Japanese Registered Patent No. 6502431 have not allowed intraocular lenses to be reused for intrascleral fixation. In addition, a problem in which the intrascleral fixation technique itself is highly challenging has been recognized.

The present disclosure provides an ophthalmic device that facilitates intrascleral fixation using an intraocular lens that has fallen into the eyeball among intrascleral fixation techniques for an intraocular lens.

An ophthalmic device for use in intrascleral fixation of an intraocular lens, according to one configuration is linear and has a longitudinal direction. The ophthalmic device includes: a sliding member configured to be slidable in the longitudinal direction where the sliding member is provided at a portion spaced apart by a predetermined distance from an end of the ophthalmic device to be punctured into an eye. An opening is formed between the end and the sliding member, the sliding member is configured to be slidable only in one direction in the longitudinal direction toward the end, and the opening is variable in size as the sliding member slides toward the end.

In the ophthalmic device according to one configuration, in the eye, a support of the intraocular lens may be inserted into the opening, and the sliding member may be slid in the eye toward the end to cause the opening to hold the support of the intraocular lens.

In the ophthalmic device according to one configuration, a portion of the opening that holds the support of the intraocular lens may include a projection.

In the ophthalmic device according to one configuration, the opening is provided by folding back a resin thread and passing both ends thereof through the sliding member.

In the ophthalmic device according to one configuration, the opening is provided by a slit formed in the longitudinal direction at an end of a resin strip.

The ophthalmic device according to one configuration may be formed such that the sliding member is configured to be slidable only in one direction toward the end by serrations provided in the longitudinal direction from the end at which the opening is located and a claw provided on the sliding member.

### Advantageous Effects of the Invention

The present disclosure provides an ophthalmic device that facilitates intrascleral fixation using an intraocular lens that has fallen into the eyeball among intrascleral fixation techniques for an intraocular lens.

### BRIEF DESCRIPTION OF THE DRAWINGS

Features, advantages, and technical and industrial significance of exemplary configurations will be described below with reference to the accompanying drawings, in which like numerals denote like elements, and wherein:

FIG. 1A shows a schematic diagram illustrating an intraocular lens, FIG. 1B shows a cross-sectional schematic diagram illustrating an eye in which an intraocular lens has been implanted,

FIG. 2 shows a schematic diagram illustrating an ophthalmic device according to one configuration,

FIGS. 3A to 3E show schematic diagrams illustrating a form of use of the ophthalmic device according to one configuration,

FIG. 4 shows a schematic diagram illustrating an ophthalmic device according to one configuration, and

FIGS. 5A to 5E show schematic diagrams illustrating a form of use of the ophthalmic device according to one configuration.

### REFERENCE SIGNS LIST

1: Intraocular Lens
2: Optical Portion
3: Support (Haptics)
6: Needle
7: Puncture Hole
8: Instrument
10: Ophthalmic Device
11: End
12: Opening
13: Sliding Member
14: Resin Thread
20: Ophthalmic Device
21: End
22: Opening
23: Sliding Member
24: Resin Strip

### DETAILED DESCRIPTION

With reference to the drawings, an example of my disclosure according to this disclosure (also referred to as this disclosure) will be described. The drawings each are an example, and the disclosure is not limited to what is illustrated in the drawings. For example, the proportions of each portion of an ophthalmic device, the size thereof relative to the eye, and the ratio thereof to instruments, or intraocular lenses, for example, are not limited to those shown in the drawings. It should be noted that the drawings are not drawn to the actual proportions for ease of understanding.

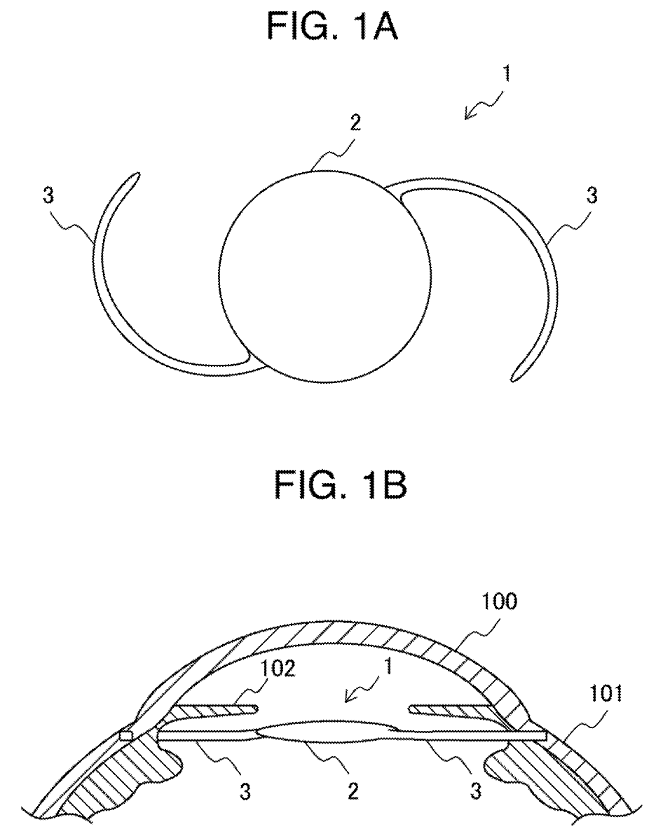

FIG. 1A shows a schematic diagram illustrating an example of an intraocular lens. The intraocular lens 1 has an optical portion 2 and supports 3. Although various types of intraocular lenses 1 may exist, the present disclosure can be applied to any type of intraocular lens 1. FIG. 1B shows a cross-sectional schematic diagram illustrating an eye with an intraocular lens placed (implanted) in the eye. FIG. 1B shows an example of an intraocular lens 1 placed in the eye by intrascleral fixation. In the intraocular lens 1, the optical portion 2 is positioned behind the iris 102 when the human gaze direction is anterior, and in the case of intrascleral fixation, the supports 3 of the intraocular lens are fixed to the sclera 101 of the eye. Although FIG. 1B shows a case in which the entire crystalline lens has been removed, other methods of attaching the intraocular lens 1 may be provided, such as removing the crystalline lens clouded by cataracts and placing and fixing the intraocular lens in the crystalline lens sac or sewing the intraocular lens 1 onto the sclera 101 with a thread.

The intraocular lens 1 placed in the eye by these methods may shift for some reason and fall into the eye. An ophthalmic device is provided that is especially suitable for fixation with an intrascleral fixation technique utilizing an intraocular lens that has fallen.

### First Configuration

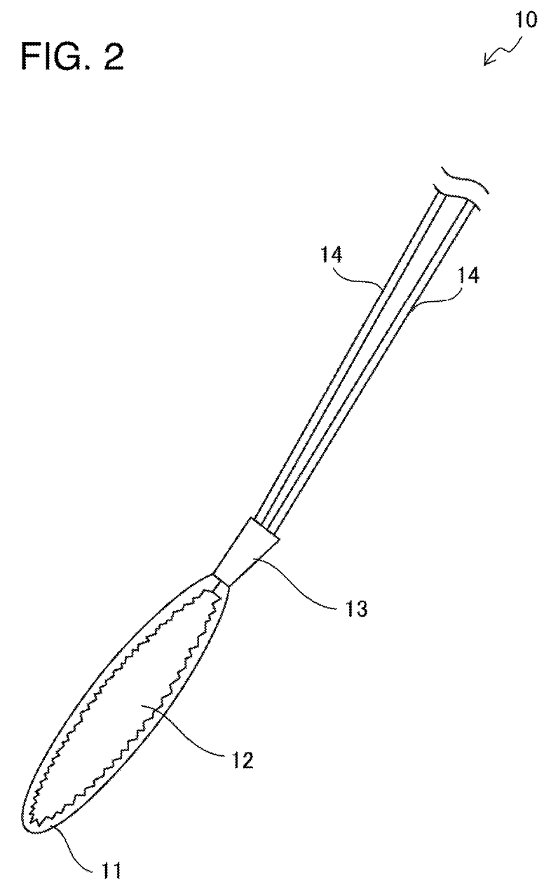

FIG. 2 shows a schematic diagram illustrating an ophthalmic device. An ophthalmic device 10 may be used for intrascleral fixation of the intraocular lens 1. As shown in FIG. 2, the ophthalmic device 10 may be linear and have a longitudinal direction. The ophthalmic device 10 may be formed from a sliding member 13 and thread 14. The ophthalmic device 10 (sliding member 13 and thread 14) may be made of resin such as acrylic, silicone, or nylon, for example. The sliding member 13 and thread 14 may be made of the same or different materials. Since the ophthalmic device 10 remains placed in the eye along with the intraocular lens 1, it can be made of the same material as that of the intraocular lens 1, for example, poly vinylidene fluoride (PVDF). The materials of the ophthalmic device 10 are not limited to these.

As described in detail below, the thickness of the ophthalmic device 10 may be within a range of, for example, 0.4 to 0.7 mm, such that it can pass through a puncture hole created by a 23- to 27-gauge needle.

The ophthalmic device 10 may have an end 11 that is punctured into the eye, a sliding member (slider) 13 that is slidable in the longitudinal direction at a predetermined distance from the end 11, and an opening 12 formed between the end 11 and the sliding member 13. The predetermined distance between the end 11 and the sliding member 13 may be a distance at which the sliding member 13 is positioned within the eye when the ophthalmic device 10 is inserted into the eye from the end 11, and may be, for example, approximately 0.3 to 1.0 mm.

In the case of the ophthalmic device 10 shown in FIG. 2, the opening 12 is provided by folding back the resin thread 14 and passing the folded end through the sliding member 13. The manufacturing methods of the ophthalmic device 10 are not limited to those described above. For example, the opening 12 may be provided by folding back the resin thread 14 and passing both ends of the thread 14 through the sliding member 13. Here, the sliding member 13 is formed to be slidable only in one direction in the longitudinal direction of the ophthalmic device 10 toward the end 11. For example, a claw may be formed on the sliding member 13 and serrations may be formed on the thread 14, and the engagement therebetween may restrict the sliding direction. Alternatively, the thread 14 and sliding member 13 may be processed to increase the friction between the thread 14 and the sliding member 13. Although described as “thread,” the member 14 may be referred to as a tube, pipe, rod, ribbon, or strip, for example, if it has an elongated shape made of flexible resin.

The opening 12 is configured to be variable in size as the sliding member 13 is slid toward the end 11. The support 3 of the intraocular lens 1 is inserted into the opening 12 as will be described in detail below. The ophthalmic device 10 is configured such that the sliding member 13 is slid toward the end 11 to reduce the aperture of the opening 12 in this state to allow the ophthalmic device 10 to hold the support 3. As shown in FIG. 2, the side of the opening 12 that holds the support 3 may be serrated or projecting to improve the retention of the support 3.

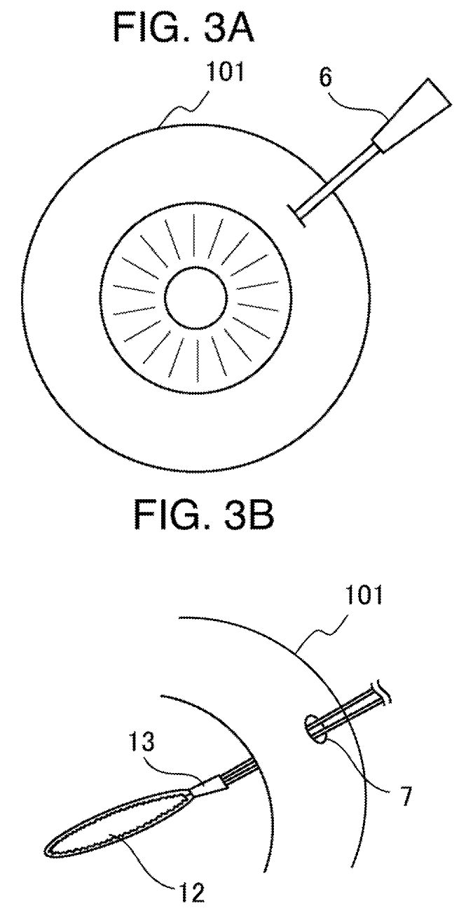

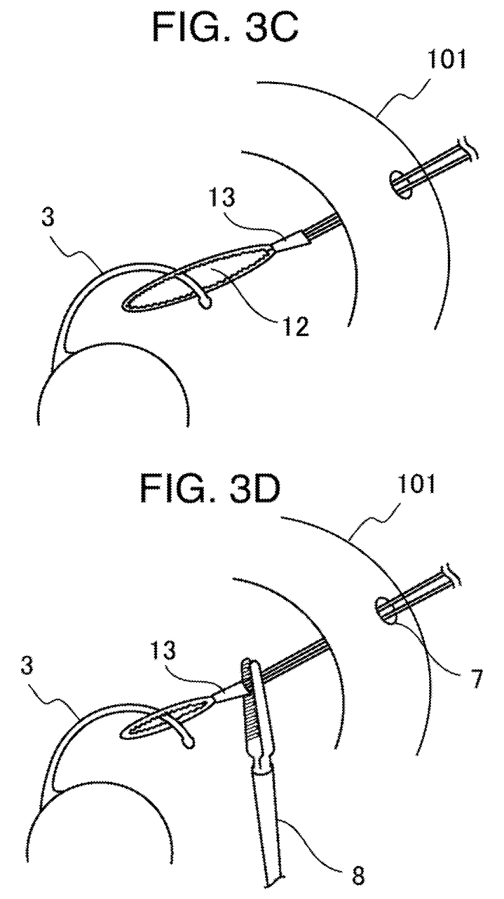

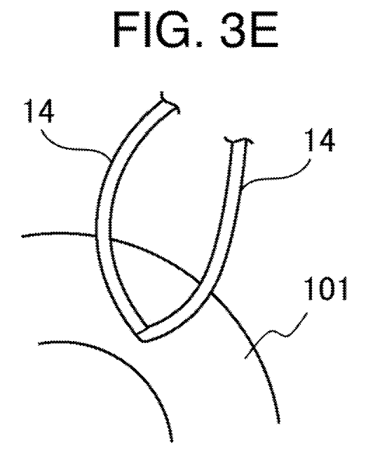

Next, with reference to FIGS. 3A - 3E, the procedure for fixing the intraocular lens 1, which has fallen into the eye, to the sclera 101 by the ophthalmic device 10 will be described. First, as shown in FIG. 3A, a needle 6 punctures the sclera 101 therethrough. The puncture site for the needle 6 may be approximately 2.0 to 2.5 mm from the cornea. Next, as shown in FIG. 3B, the ophthalmic device 10 is inserted into the eye through the puncture hole 7 by the needle 6. From FIG. 3B onward, the cornea is not shown in the drawings, but a transilluminated view of the inside of the eye is shown. Next, as shown in FIG. 3C, the support 3 of the intraocular lens 1 in the eye is inserted into the opening 12 by the surgeon. The instruments operated by the surgeon may be inserted through a different puncture hole. From this state, as shown in FIG. 3D, the sliding member 13 is slid toward the end 11 by the surgeon using the instrument 8, the opening 12 is closed, and the support 3 of the intraocular lens 1 is held by the ophthalmic device 10. The other support 3 of the intraocular lens 1 is then also held in the same manner by the ophthalmic device 10 inserted from the opposite side, following the procedure in FIGS. 3A - 3D. The positions of both the ophthalmic devices 10 (i.e., the position of the intraocular lens 1) may then be adjusted within the eye. After completion of positioning, the remaining thread 14 outside the sclera 101 may be ligated and further cut and embedded within the sclera 101 as shown in FIG. 3E. The method of processing the thread 14 is not limited to this if the ophthalmic device 10 can be fixed.

As described above, according to the ophthalmic device of one configuration of this disclosure, an intraocular lens can be held by the procedure of inserting each support of the intraocular lens into the opening and holding it by reducing the size of the opening. This facilitates intrascleral fixation of the intraocular lens in the eye.

### Second Configuration

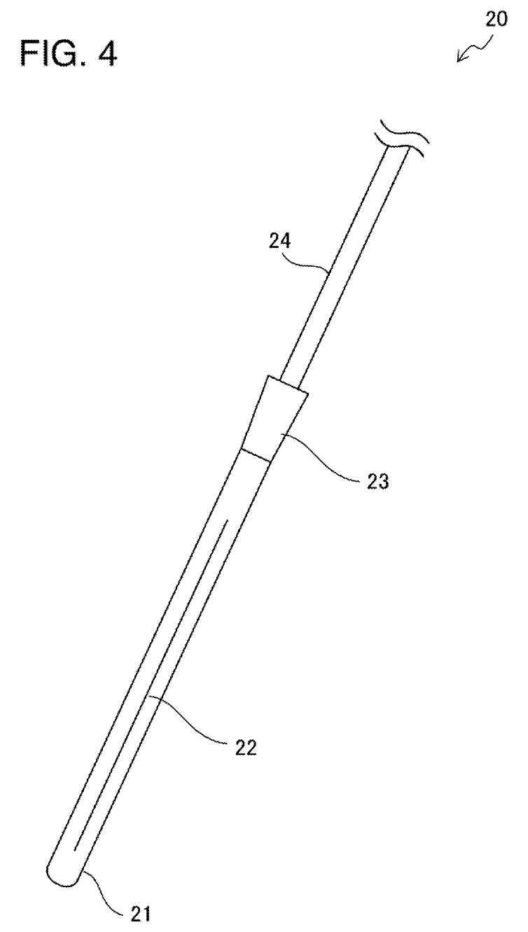

FIG. 4 shows a schematic diagram illustrating an ophthalmic device according to another configuration of this disclosure. As shown in FIG. 4, the ophthalmic device 20 may be linear and have a longitudinal direction in the same manner as in the ophthalmic device 10. The ophthalmic device 20 may be formed from a sliding member 23 and strip 24. In the second configuration, the ophthalmic device 20 (sliding member 23 and strip 24) may also be made of resin such as acrylic, silicone, or nylon, for example. The sliding member 23 and strip 24 may be made of the same or different materials. Since the ophthalmic device 20 is placed in the eye along with the intraocular lens 1, it can be made of the same material as that of the intraocular lens 1, for example, poly vinylidene fluoride (PVDF). The materials of the ophthalmic device 20 are not limited to these.

In the same manner as in the ophthalmic device 10, the ophthalmic device 20 as a whole also has a thickness that allows it to pass through a puncture hole formed by a 23- to 27-gauge needle, and may have, for example, a thickness of 0.4 to 0.7 mm or less.

The ophthalmic device 20 may have an end 21 that is punctured into the eye, a sliding member 23 that is slidable in the longitudinal direction at a predetermined distance from the end 21, and an opening 22 formed between the end 21 and the sliding member 23. The predetermined distance between the end 21 and the sliding member 23 may be the same as the predetermined distance between the end 11 and the sliding member 13 in the first configuration.

In the case of the ophthalmic device 20 shown in FIG. 4, the opening 22 is provided at the end of the resin strip 24 by a slit in the longitudinal direction. That is, this slit serves as the opening 22 when it is opened by the surgeon during the surgery. The ophthalmic device 20 may be manufactured by passing the end having the slit through the sliding member 23. The manufacturing methods of the ophthalmic device 20 are not limited to those described above. Here, the sliding member 23 is formed to be slidable only in one direction in the longitudinal direction of the ophthalmic device 20 toward the end 21. This may also be configured in the same manner as, for example, the sliding member 13 and thread 14 of the ophthalmic device 10. Although described as a “strip,” the member 24 may be referred to as a strip, strap, ribbon, tape, or band, for example, if it is made of flexible resin and has an elongated shape with a width.

The opening 22 is configured to be variable in size as the sliding member 23 is slid toward the end 21. In the same manner as in the ophthalmic device 10, in the ophthalmic device 20, which will be described in detail later, the support 3 of the intraocular lens 1 is inserted into the opening 22 as well. The ophthalmic device 20 is configured such that the sliding member 23 is slid toward the end 21 to reduce the aperture of the opening 22 in this state to allow the ophthalmic device 20 to hold the support 3.

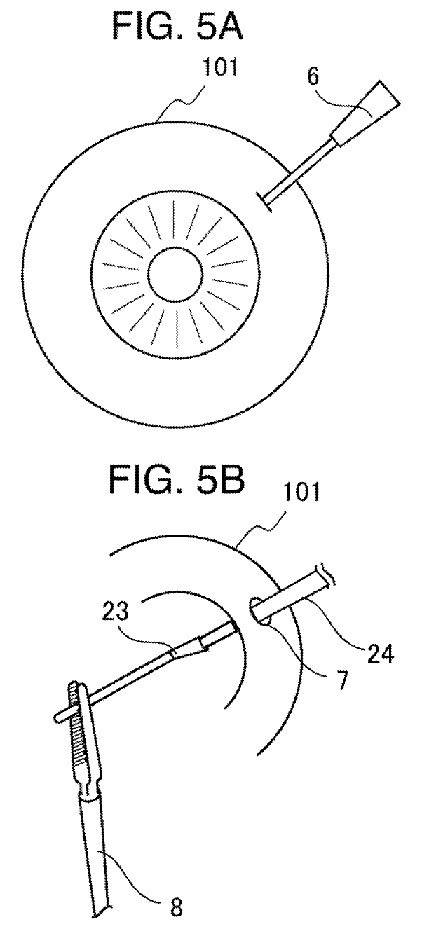

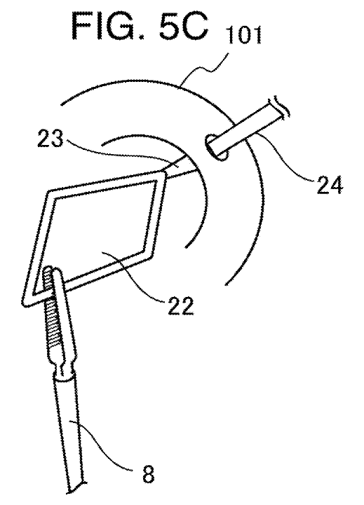

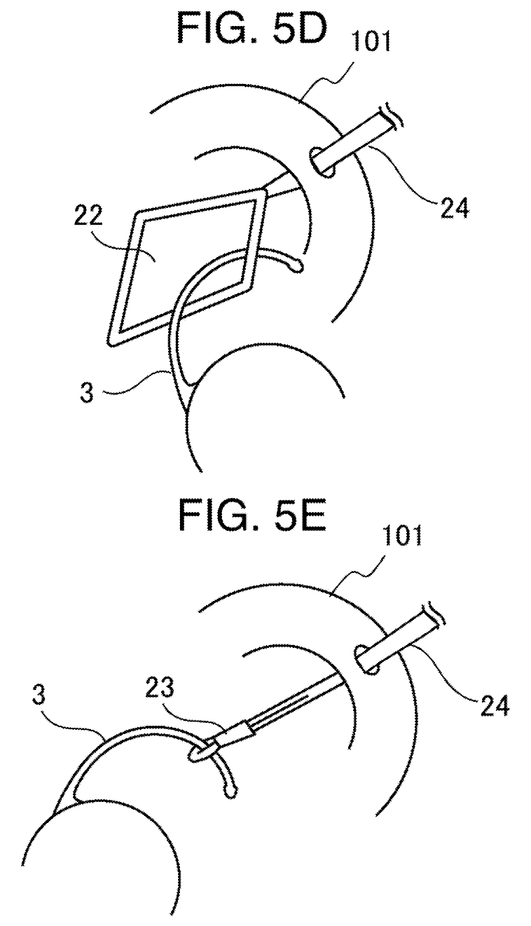

Next, with reference to FIG. 5, the procedure for intrascleral fixation with the ophthalmic device 20 will be described. First, as shown in FIG. 5A, a needle 6 punctures the sclera 101 therethrough. This may be the same as described for the ophthalmic device 10 with reference to FIG. 3A, and thus is omitted. Next, as shown in FIG. 5B, the ophthalmic device 20 is inserted into the eye through the puncture hole 7 by the needle 6. From FIG. 5B onward, the cornea is not shown in the drawings, but a transilluminated view of the inside of the eye is shown. Next, as shown in FIG. 5C, the opening 22 of the ophthalmic device 20 in the eye is opened by the surgeon using the instrument 8. The instrument 8 operated by the surgeon may be inserted through another puncture hole. Here, in the example in FIG. 5C, the shape of the opening 22 is shown as a rhombus, but the shape of the opening 22 is not limited to this and may be an oval.

Next, as shown in FIG. 5D, the support 3 of the intraocular lens 1 in the eye is inserted into the opening 22 by the surgeon. From this state, as shown in FIG. 5E, the sliding member 23 is slid toward the end 21 by the surgeon, the opening 22 is closed, and the support 3 of the intraocular lens 1 is held by the ophthalmic device 20. The other support 3 of the intraocular lens 1 is then also held in the same manner by the ophthalmic device 20 inserted from the opposite side, following the procedure in FIGS. 5A- 5D. The positions of both the ophthalmic devices 20 (i.e., the position of the intraocular lens 1) may then be adjusted within the eye. After completion of positioning, the strip 24 that remains outside of the sclera 101 may be cut, and the end is thermally processed to be embedded within the sclera 101. The method of processing the strip 24 is not limited to this if the ophthalmic device 20 can be fixed.

As described above, according to the ophthalmic device of another configuration of this disclosure, an intraocular lens can also be held by the procedure of inserting each support of the intraocular lens into the opening and holding it by reducing the size of the opening. This facilitates intrascleral fixation of the intraocular lens in the eye.

Although this disclosure has been described on the basis of the drawings and configurations, it should be noted that one skilled in the art can easily make various changes and modifications on the basis of the present disclosure. Accordingly, it should be noted that these changes and modifications are included in the scope of this disclosure.

For example, the intrascleral fixation of the intraocular lens that has fallen into the eye is described above. However, the ophthalmic device may also be applicable to a case where a new intraocular lens is inserted and attached from outside the eye.

What is claimed is:

1. An ophthalmic device for use in intrascleral fixation of an intraocular lens, the ophthalmic device being linear and having a longitudinal direction, comprising:
a sliding member configured to be slidable in the longitudinal direction, the sliding member being provided at a portion spaced apart by a predetermined distance from an end of the ophthalmic device to be punctured into an eye, wherein
an opening is formed between the end and the sliding member,
the sliding member is configured to be slidable only in one direction in the longitudinal direction toward the end, and
the opening is variable in size as the sliding member slides toward the end.

2. The ophthalmic device according to claim 1, wherein
in the eye, a support of the intraocular lens is inserted into the opening, and
the sliding member is slid in the eye toward the end to cause the opening to hold the support of the intraocular lens.

3. The ophthalmic device according to claim 2, wherein a portion of the opening that holds the support of the intraocular lens includes a projection.

4. The ophthalmic device according to claim 1, wherein the opening is provided by folding back a resin thread and passing both ends thereof through the sliding member.

5. The ophthalmic device according to claim 1, wherein the opening is provided by a slit formed in the longitudinal direction at an end of a resin strip.

6. The ophthalmic device according to claim 1, wherein the sliding member is configured to be slidable only in one direction toward the end by serrations provided in the longitudinal direction from the end at which the opening is located and a claw provided on the sliding member.
